(19) United States
(12) Patent Application Publication
(10) Pub. No.: US 2015/0250714 A1
(43) Pub. Date: Sep. 10, 2015

(54) PHARMACEUTICAL COMPOSITIONS AND DELIVERY DEVICES COMPRISING STINGING CELLS OR CAPSULES

(71) Applicant: NANOCYTE (ISRAEL) LTD., Caesarea (IL)

(72) Inventors: Shimon Eckhouse, Haifa (IL); Tamar LOTAN, Moshava Kineret (IL); Ari AYALON, Haifa (IL)

(73) Assignee: NANOCYTE (ISRAEL) LTD., Caesarea (IL)

(21) Appl. No.: 14/717,061

(22) Filed: May 20, 2015

Related U.S. Application Data
(62) Division of application No. 13/882,190, filed on Apr. 28, 2013.
(60) Provisional application No. 61/407,073, filed on Oct. 27, 2010.

(51) Int. Cl. A61K 9/00; A61K 35/614; A61K 31/46

(52) U.S. Cl. CPC A61K 9/0021; A61K 31/46; A61K 35/614

(57) ABSTRACT

A pharmaceutical composition is provided. The pharmaceutical composition comprises as an active ingredient a tropane alkaloid drug or a muscarinic receptor antagonist and stinging cells or capsules.

### RELATED APPLICATIONS

This application is a division of U.S. patent application Ser. No. 13/882,190 filed on Apr. 28, 2013, which is a National Phase of PCT Patent Application No. PCT/IL2011/000831 having International Filing Date of Oct. 27, 2011, which claims the benefit of priority under 35 USC §119(e) of U.S. Provisional Patent Application No. 61/407,073 filed on Oct. 27, 2010. The contents of the above applications are all incorporated by reference as if fully set forth herein in their entirety

### FIELD AND BACKGROUND OF THE INVENTION

The present invention, in some embodiments thereof, relates to pharmaceutical compositions and delivery devices comprising stinging cells or capsules.

The advantage of transdermal delivery of hydrophilic drugs versus oral delivery lies in the molecular nature of the GI tract. As a lipid membrane, the GI tract possesses hydrophobic properties, thus the more hydrophilic a drug is, the more likely it is to absorb poorly through the GI tract. A well-known example of this problem is sodium alendronate, a bisphosphonate that needs to be administered in very large doses because only 0.64% is absorbed when taken orally. This problem of poor absorption can sometimes lead to situations in which a hydrophilic drug has been developed, it then becomes necessary to develop a complementary prodrug that will increase absorption in the GI tract in order to make the drug effective. This prodrug increases both the cost of the drug and may carry side effects of its own. There have even been cases in which development ceased on a drug because the problem of poor absorption could simply not be overcome. In other cases, hydrophilic drugs that absorb poorly must be injected, causing patient discomfort.

Delivery of hydrophilic drugs through a transdermal patch presents a solution to many of the problems currently associated with other delivery methods for hydrophilic drugs, most notably, by avoiding all GI tract issues. However, owing to the hydrophobic nature of the skin and its role as a bather for keeping unwanted substances out of the body, there have been challenges in this approach. Until recently, for transdermal delivery of hydrophilic drugs to be effective, it was necessary to use an active delivery system to breech the skin barrier, thereby allowing the drugs to absorb.

A small subset of enhancers that increase skin permeability without irritation have been used successfully to deliver small molecules, but have had limited impact on the problem of delivering hydrophilic compounds or macromolecules. Overall, chemical enhancers can increase skin permeability and provide an added driving force for transport by increasing drug partitioning into the skin (thereby increasing the concentration gradient driving diffusion), but the difficulty of localizing their effects to the stratum corneum so as to avoid irritation or toxicity to living cells in the deeper skin has severely constrained their application.

While transdermal delivery of hydrophilic drugs does appear to offer solutions to some common problems, it is important to remember that transdermal delivery is not without some challenges. Today, these challenges range from medical limitations to cosmetic concerns. To begin with, the existing patches on the market are limited to small molecular weight drugs with very small daily dosages. In addition, the place that the patch is used must be changed to avoid irritating the skin, meaning that the same spot on the skin cannot be used over and over again.

The phylum Cnidaria, consisting of sea anemones, corals, jellyfish and hydra, is one of the most ancient multicellular phyla, typically featuring stinging cells containing cnidocysts, microcapsules equipped with an injection system (3). Cnidocyst content is composed of a folded thin tubule embedded in a matrix of high concentration of poly γ glutamate aggregate trapped with cationic metal (4). Upon activation, water influx increases the intracapsular osmotic pressure to 150 bars resulting in an immediate discharge and injection of the long thin tubule at an ultrafast acceleration of 5*10⁶ G (5). About 30 subtypes of cnidocysts, differing in size and shape are known, but all function by the same basic principle. The use of stinging capsules for (trans/intra) dermal delivery of active ingredients and cosmetics have been previously suggested (e.g., WO02/26191).

### SUMMARY OF THE INVENTION

According to an aspect of some embodiments of the present invention there is provided a pharmaceutical composition comprising as an active ingredient a tropane alkaloid drug and stinging cells or capsules.

According to an aspect of some embodiments of the present invention there is provided a pharmaceutical composition comprising as an active ingredient a muscarinic receptor antagonist and stinging cells or capsules.

According to some embodiments of the invention, the active ingredient is disposed in a liquid surrounding, or stored within, the stinging cells or capsules.

According to an aspect of some embodiments of the present invention there is provided a delivery device comprising a support which serves for supporting the stinging cells or capsules of the pharmaceutical composition and for applying it to an outer surface of a tissue region into which delivery is desired.

According to some embodiments of the invention, the tropane alkaloid drug is scopolamine.

According to some embodiments of the invention, the muscarinic receptor antagonist is scopolamine.

According to some embodiments of the invention, the muscarinic receptor antagonist is atropine.

According to some embodiments of the invention, the tropane alkaloid drug is selected from the group consisting of scopolamine, atropine hyoscyamine, cocaine, catuabine and phenyltropane.

According to some embodiments of the invention, the muscarinic receptor antagonist is a hydrophilic drug.

According to some embodiments of the invention, the muscarinic receptor antagonist is selected from the group consisting of scopolamine, atropine, hyoscyamine, ipratropium, tropicamide, pirenzepine, diphenhydramine, diminhydrinate, dicyclomine, flavoxate, oxybutynin, tiotropium, cyclopentolate, atropine methonitrate, trihexyphenidyl, tolterodine, solifenacin, darifenacin, benzatropine, mebeverine and procyclidine.

According to some embodiments of the invention, the pharmaceutical composition further comprises a pharmaceutically acceptable carrier selected from the group consisting of an aqueous solution, a gel, an oil and semi solid formulation.

According to some embodiments of the invention, the stinging capsules are capable of delivering upon discharge the liquid disposed in or around the stinging capsules into a tissue.

According to some embodiments of the invention, the device further comprises a mechanism for triggering the discharge of the stinging capsules or cells.

According to some embodiments of the invention, the mechanism is selected from the group consisting of a chemical triggering mechanism and an electrical triggering mechanism.

According to some embodiments of the invention, the support is selected from the group consisting of a patch, a foil and a plaster.

According to some embodiments of the invention, the tissue region is a skin.

According to some embodiments of the invention, the stinging cells or capsules are from an organism selected from the group consisting of Aiptasia diaphana, Aiptasia mutabilis Aiptasia pallida, hydra vulgaris, Rhopilema nomadica, Nematostella vectensis and Pelagia nuctiluca.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C are photographs showing the cnidocysts, and the discharged capsules. FIG. 1A-A filament of Aiptasia diaphana containing packed cnidocysts, bar-100 microns. FIG. 1B-A preparation of isolated cnidocysts of 60 microns in length, 8 microns in diameter and with a folded in 150 micron injector, bar-25 microns. FIG. 1C-A discharged microcapsule the arrow point to the thin tubule that penetrates the skin, bar=25 micron.

FIGS. 2A-C are photographs showing in-vitro (FIGS. 2A and 2B) and in-vivo (FIG. 2C) delivery of a hydrophilic compound by nematocysts formulation (FIG. 2A) 5-min activation of nematocysts over porcine ear skin with 0.05% toulidine blue solution. Black arrow, penetrating cnidocyst. White arrow, skin hair bundle. (FIG. 2B) The skin was tape-stripped 30 times to remove the stratum corneum and photographed. Scale bar (A,B) 100 micron. (FIG. 2C) Scopolamine levels in pig plasma after topical exposure of 5 min Test group (closed squared, ▪) containing gel formulation with isolated cnidocysts and control group (open triangles, Δ) containing gel formulation only. Test and control groups were both treated with the same solution of scopolamine. Data are shown as mean+SD;

FIGS. 3A-B are graphs showing the mean plasma concentration over time of atropine after IM and topical administration of 0.05 mg/kg and 400 mg atropine, respectively (pigs 1483-topical-session1 and 1461-IM-session1 were excluded, the time point 5 min obtained in pig-1498-S1-topical was not taken into account).

### DESCRIPTION OF SPECIFIC EMBODIMENTS OF THE INVENTION

The present invention, in some embodiments thereof, relates to pharmaceutical compositions and delivery devices comprising stinging cells or capsules.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Delivery of hydrophilic drugs through a transdermal patch presents a solution to many of the problems currently associated with other delivery methods for hydrophilic drugs, most notably, by avoiding all GI tract issues. However, owing to the hydrophobic nature of the skin and its role as a barrier for keeping unwanted substances out of the body, there have been challenges in this approach. The development of effective transdermal delivery systems for hydrophilic drugs should increase the number of hydrophilic drugs available to patients. These systems will also make it easier for pharmaceutical companies to develop and bring new hydrophilic drugs to market because they will no longer need to worry about the problems caused by oral delivery.

All members of the phylum Cnidaria possess a sophisticated injection system folded within cnidocysts. Upon cnidocyst activation high internal pressure develops which triggers the ultrafast discharge of a long thin tubule that penetrates the prey and injects the cnidocyst content.

The present inventors have now shown, the feasibility of using a topically applied gel containing isolated cnidocysts for immediate systemic delivery of hydrophilic drugs such as the muscarinic antagonist, tropane alkaloid, scopolamine and atropine. The system is compatible with hydrophilic drugs and its safety has been clinically tested. The ability of the isolated, formulated injector to penetrate a barrier as thick as the skin presents an exciting alternative for transdermal drug delivery.

Thus, according to an aspect of the invention there is provided a pharmaceutical composition comprising, as an active ingredient, a tropane alkaloid drug and stinging cells or capsules.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the tropane alkaloid or muscarinic antagonist accountable for the biological effect.

As used herein a “tropane alkaloid drug” refers to are a class of alkaloids and secondary metabolites that contain a tropane ring in their chemical structure. Tropane alkaloids occur naturally in many members of the plant family Solanaceae. Tropane alkaloid molecules according to the present teachings refer both to naturally occurring and synthetically synthesized (i.e., synthetic) molecules. The tropane alkaloid drug may be (but not limited to) an anticholihergic drug or a stimulant.

Non-limiting examples of anticholinergic drugs and deliriants are provided infra:

Atropine, racemic hyoscyamine, from the deadly nightshade (Atropa belladonna)

Hyoscyamine, the levo-isomer of atropine, from henbane (Hyoscamus niger) and mandrake (Mandragora officinalis)

Scopolamine, from henbane and Datura species (Jimson weed)

All three ACh-inhibiting (Acetylcholine) chemicals can be found in the leaves, stems, and flowers in varying, unknown amounts in the tree-cousin of Datura, (Brugmansiae) more commonly known as Angel Trumpets or Devil's Weed.

Stimulants:

Stimulants and cocaine-related alkaloids:

Cocaine, from Erythroxylum coca

Ecgonine, a precursor and metabolite of cocaine

Benzoylecgonine, a metabolite of cocaine

Hydroxytropacocaine, from Erythroxylum coca

Methylecgonine cinnamate, from Erythroxylum coca

According to a specific embodiment the tropane alkaloid is a Catuabine used in the preparation of catuaba (as an aphrodisiac).

According to an additional aspect of the invention there is provided a pharmaceutical composition comprising as an active ingredient a muscarinic receptor antagonist and stinging cells or capsules.

As used herein a “muscarinic receptor antagonist” refers a molecule that reduces the activity of the muscarinic acetylcholine receptor. The molecule may be naturally occurring or synthetically produced (i.e., synthetic). According to a specific embodiment the molecule is hydrophilic.

Below in Table 1, are non-limiting examples of such molecules.

TABLE 1

| | | Trade | |
|---|---|---|---|
| Clinical use | Mechanism | names | Substance |
| in anaesthesia | non-selective antagonism, CNS stimulation | | Atropine (D/L- |
| anticholinesterase | | | Hyoscyamine) |
| poisoning | | | |
| bradycardia | | | |
| antispasmodic | | | |
| in | | | |
| gastrointestinal | | | |
| hypermotility | | | |
| as atropine | non-selective antagonism, CNS depression | Scopace, | Scopolamine (L- |
| motion | | Transderm- | Hyoscine) |
| sickness | | Scop, | |
| | | Maldemar, | |
| | | Buscopan | |
| in asthma and | non-selective antagonism, without any mucociliary | Atrovent | Ipratropium |
| bronchitis | excretion inhibition. | and | |
| | | Apovent | |
| produce | short acting non-selective antagonism, CNS | | Tropicamide |
| mydriasis and | depressionwww(dot)en(dot)wikipedia(dot)org/wiki/ | | |
| cycloplegia in | Muscarinic_antagonist - cite_note-Rang147- | | |
| diagnostics | 0#cite_note-Rang147-0 | | |
| in peptic ulcer | M1 receptor-selective antagonist | | Pirenzepine |
| | inhibits gastric secretion | | |
| for | | Benadryl | Diphenhydramine |
| extrapyramidal | | | |
| symptoms | | | |
| from typical | | | |
| antipsychotic | | | |
| medications | | | |
| antihistamine | | | |
| sleep aid | | | |
| motion | | Dramamine | Dimenhydrinate |
| sickness | | | |
| | | | Dicyclomine |
| | | | Flavoxate |
| overactive | | Ditropan | Oxybutynin |
| bladder | | | |
| urge | | | |
| incontinence | | | |
| Chronic | | Spiriva | Tiotropium |
| obstructive | | | |
| pulmonary | | | |
| disease | | | |
| produce | short acting non-selective antagonism, CNS | | Cyclopentolate |
| mydriasis and | depression | | |
| cycloplegia in | | | |
| diagnostics | | | |
| antispasmodic | non-selective antagonism, blocks transmission in | | Atropine |
| in | ganglia. Lacks CNS effects | | methonitrate |
| gastrointestinal | | | |
| hypermotility | | | |
| Parkinson's disease | Targets the M1 Muscarinic receptor | Artane | Trihexyphenidyl/ |
| | | | Benzhexol |
| | | Detrusitol, | Tolterodine |
| | | Detrol | |
| overactive | Competitive muscarinic acetylcholine receptor | Vesicare | Solifenacin |
| bladder | antagonist | | |
| (OAB) | | | |
| Urgency (urge | | | |
| incontinence) | | | |
| Urinary incontinence | Selective for M3 receptors | Enablex | Darifenacin |
| | www(dot)en(dot)wikipedia(dot)org/wiki/ | | |
| | Muscarinic_antagonist - | | |
| | cite_note-Rang(dot)26Dale6th10- | | |
| | 5-2#cite_note-Rang(dot)26Dale6th10-5-2 | | |
| Parkinson's disease | Reduces the effects of the relative central | Cogentin | Benzatropine |
| | cholinergic excess that occurs as a result of | | |
| | dopamine deficiency. | | |
| Irritable bowel | A muscolotropic spasmolytic with a strong and | Colofac, | Mebeverine |
| syndrome in | selective action on the smooth muscle spasm of | Duspatal, | |
| its primary | the gastrointestinal tract, particularly of the colon. | Duspatalin | |
| form (e.g. | | | |
| Abdominal | | | |
| Pain, Bloating | | | |
| Constipation, | | | |
| and Diarrhea). | | | |
| Irritable bowel | | | |
| syndrome | | | |
| associated | | | |
| with organic | | | |
| lesions of the | | | |
| gastrointestinal | | | |
| tract. (e.g. | | | |
| diverticulosis & | | | |
| diverticulitis, | | | |
| etc.). | | | |
| Drug-induced | Antimuscarinic | | Procyclidine |
| parkinsonism, | | | |
| akathisia and | | | |
| acute | | | |
| dystonia; | | | |
| Parkinson | | | |
| disease; and | | | |
| Idiopathic or | | | |
| secondary | | | |
| dystonia | | | |

According to a specific embodiment, the molecule is scopolamine.

According to a specific embodiment, the molecule is atropine.

As used herein the phrase “stinging capsules” refers to the capsules (cnidocysts), which are contained in stinging cells. The phrase “stinging cells” refers to the specialized cells (e.g. cnidocytes or nematocytes) present in, for example, all members of the phylum Cnidaria, Myxozoa, and Dinoflagellata. The stinging capsules house the delivery tubule. The stinging capsules act as microscopic syringes and serve as a prey or defense mechanism. The stinging capsule is a hardened dense capsule filled with liquid, containing a highly folded inverted tubule which sometimes features specialized structures such as shafts, barbs, spines, and/or stylets.

The stinging capsule according to the teachings of the present invention can be an isolated stinging capsule or alternatively it can form a part of a stinging cell.

In any case, the stinging capsule or cell is derived from an organism of the phylum Cnidaria, Myxozoa, or Dinoflagellata.

The stinging cell or capsule utilized by the present invention is preferably derived from an organism of the class Anthozoa, Hydrozoa or Scyphozoa. More specifically, the stinging cell/capsule utilized by the present invention can be derived from, for example, subclasses Hexacorallia or Octocorallia of the class Anthozoa, (mostly sea anemone and corals), subclasses Siponophora or Hydroida of the class Hydrozoa, or from subclasses Rhisostomeae or Semastomeae of the class Scyphozoa.

According to a specific embodiment the stinging cell/capsule is derived from a sea anemone. According to a specific embodiment the sea anemone is Nematostella vectensis.

Stinging capsules from such organisms include toxins, which are non-toxic to humans, and other mammals. As such, these stinging cells or capsules isolated therefrom are ideally suited for safe and efficient delivery of the active agent into mammalian tissue.

It will be appreciated that the use of stinging cells from organisms which sequester toxins that are not fatal but cause only minor irritations to, for example, mammals, is also envisioned by the present invention.

In addition, stinging cells from other sources can also be utilized by the present invention provided inactivation of the endogenous toxin is effected prior to use.

Such inactivation can be effected via one of several methods, including but not limited to, temperature or chemical denaturation, enzymatic inactivation or ligand inactivation (e.g., Fab fragment of an antibody).

As is demonstrated in U.S. Pat. No. 6,613,344, toxins endogenous to cnidocysts can be efficiently and easily inactivated by incubating isolated cnidocysts at 45° C. for several hours. Alternatively, incubation at a high temperature of 70-95° C. for several minutes can also be utilized by the present invention.

As demonstrated in U.S. Pat. No. 6,613,344, incubation of cnidocysts at 45° C. for 22 hours does not damage or trigger activation of the cnidocyst. Such conditions are effective in denaturing polypeptides stored within the cnidocyst, such as the polypeptide toxins and enzymes delivered by the tubule of the cnidocyst. It will be appreciated that since organisms of, for example, the phylum Cnidaria habitat aquatic environments, which are characterized by temperatures well below 30° C., polypeptides stored within their stinging capsules can be denatured via incubation in temperatures well above 30° C.

The stinging cell or the stinging capsule of the present invention can be isolated from a cell extract prepared from organs (e.g., tentacles) or parts of an organism, which contain the stinging cells.

The main differences between the stinging cells are in their capsule shape and size and in their tubule dimensions. Examples of species containing different tubules include but are not limited to Rhopilema nomadica (400 μm hollow tubule length with tiny hollow barbs), Hydra vulgaris, Hydra hymanae, Metridium senile (200 μm tubule length), Nematostella vectensis (200 μm tubule length), Rhodactis rhodostoma (9 mm tubule length), Heliofungia actiniformis (1000 μm tubule length) and Aiptasia diaphana (150 μm tubule length).

Once isolated, the nematocysts can be concentrated from a homogenate of stinging cells via centrifugation, and freeze-dried to produce a fine powder or formulated in a gel, as described in the Examples section which follows.

According to a specific embodiment, the active ingredient (i.e., tropane alkaloid drug or muscarinic antagonist) can be disposed in or around the stinging cell/capsule. According to one preferred embodiment of the present invention, the active ingredient is disposed within the liquid stored in the stinging cell or the stinging capsule. In such a case, the stinging cell or the isolated capsule is loaded with the active ingredient via any one of several methods generally known in the art such as, but not limited to, diffusion, electroporation, liposome fusion, microinjection and the like.

Alternatively and according to another embodiment of the present invention, the active ingredient is disposed in a liquid surrounding the stinging cell or capsule. In such a case, the stinging capsule's natural mechanism of osmotically collecting liquid from the environment following triggering pumps the active ingredient into the stinging cell just prior to or during the discharge.

Yet alternatively, the active ingredient is absent from the stinging capsule or cell and is applied only following puncture of the skin with the stingling cells or capsules as is further described hereinbelow (i.e., preconditioning). This embodiment illustrates a two-component composition which is packed is separate containers. One container comprising the stinging cells/capsules and a separate, second, container which comprises the active ingredient.

In any case, since a stinging capsule is highly permeable to water and molecules, active ingredient loading prior to or during discharge can be easily achieved.

The compositions of the present invention may further comprise an orientation and proximity agent.

The orientation and proximity agent is selected for positioning at least one stinging capsule/cell in intimate proximity for applying the active ingredient to an outer surface of a tissue region (e.g., skin) into which delivery is desired and orientating it such that the opening tip of the stinging capsule (termed the operculum) from where the tubule discharges substantially faces the skin.

As used herein, the phrase “in intimate proximity with the substance” refers to a range of distances from touching the substance to the furthest distance from the substance that the tubules are still able to penetrate.

As described in U.S. patent application Ser. No. 11/108,662, the stinging capsules have asymmetric charge/structure characteristics, such that the opening tip is positively charged and the opposite end is not. Thus, by using specific agents capable of binding to the opening tip on one hand and the outer surface of the skin on the other, the capsules can be orientated so that a high proportion of stinging cells come into physical contact with the skin, thereby enhancing subsequent delivery of an active agent. Specifically, the opening tip is orientated, such that it substantially faces the skin.

Preferably, the orientation and proximity agent is a negatively charged polymer, or at least partially negatively charged, which interacts with the positively charged opening tip. Examples of negatively charged polymers that may be used in the present invention include, but are not limited to synthetic anionic polymers such as polyacrylic acid (PAA), poly saccharines, such as alginic Acid, Na alginate, hydroxy propyl methyl cellulose, carboxy methyl cellulose, and others poly saccharines such as gum karaya, gum tragacanth, poly ethylene oxide, poly vinyl alcohol, starch, lecithin. The orientation and proximity agent can be amphoteric for example gelatin, caseine and lecitin. The orientation and proximity agent may also serve as an adhesive or bioadhesive e.g. PAA, carbomer, lectin, bacterial fimbrins and invasins. The capsules can be pretreated with the orientation and proximity agents. Alternatively, the orientation and proximity agents can be introduced into the composition in which the capsules are formulated.

The compositions of the present invention may alternatively or further comprise conditioning agents which increase the porosity of the skin.

The compositions of the present invention are typically applied topically onto the skin e.g. in a gel (see the Examples section which follows). In this way, the number of tubules need not be limited to one particular area of the substance as is the case with a chip or an array, but may be spread over larger areas.

Alternatively, as described herein below, for more accurate dosing of an active agent, the stinging capsules can be applied onto the skin using an applicator such as a patch, a cap, a foil, a plaster, a polymer or a pad which can be prepared with an exact quantity of stinging capsules.

The pharmaceutical compositions described herein may further comprise carriers which stabilize the stinging cells/capsules and possibly enhance triggering efficiency. The carrier generally should not affect the ability of the stinging cells to discharge following triggering. Examples of pharmaceutical compositions suitable for topical applications include, but are not limited to powders, gels, creams, ointments, pastes, lotions, milks, suspensions, foams and serums.

As mentioned, the stinging cells/capsules described above can also be utilized in a delivery device useful for delivering the active ingredient into a tissue region of an individual. Such a device includes a support, which serves for supporting the stinging cells/capsules and for applying the stinging cells/capsules to an outer surface of the tissue region into which delivery is desired (e.g., skin). The device can be utilized to deliver the active ingredient, which is loaded into the stinging cells/capsules or disposed in a liquid surrounding stinging cells/capsules. Alternatively, the capsules or liquid surrounding same are absent of any active ingredient. According to this specific embodiment the device is applied onto the skin and discharge is effected. soon thereafter once the skin is preconditioned (i.e., open channels exist) the active ingredient is applied. Such a configuration is described in details in U.S. Patent Application No. 20060234203.

The support can be, for example, a patch, a foil, a plaster or a film or any material capable of supporting stinging cells/capsules in a manner suitable for application to, for example, a skin region of the individual.

Stinging cells/capsules can be secured to the support via, for example, biological glue (e.g. BIOBOND™), polylysine, a mesh support or the like.

Discharge of the stinging cells/capsules can be activated upon contact with tissue as described above. For example, following application of the device, pressure can be exerted on the support thus forcing contact between the stinging cells and the tissue region thereby activating discharge. Alternatively, discharge can be activated by a mechanism included within the device.

The mechanism can be an electrical or chemical activating mechanism which when activated by a physician or by the individual to be treated, triggers simultaneous discharge of the stinging cells/capsules preferably in a rapid and uniform manner.

As used herein the phrase “triggering a discharge” refers to the activation of at least one stinging capsule/cell whereby the tubule contained within is released and penetrates the skin. This may be triggered by the active agent itself or by a second agent either by hydration, a change in pH, by a biochemical change (e.g. an enzyme) or by a chemical change.

Chemical triggering can be mediated by substances such as free and conjugated N-acetylated sugars or low molecular weight amino compounds which are known to be detected by at least two classes of stinging cell chemoreceptors. Sodium thiocyanate (NaSCN) is capable of triggering discharge of cnidocysts.

In addition, Lubbock and Amos [Nature, 290(5806), 500-1, 1981] have shown that isolated cnida (cnidocysts) can discharge normally when placed in buffered EGTA or 10 mM citrate solution; Weber [Eur J Biochem, 184(2), 465-76 (1989)] demonstrated the effect of dithioerthritol or proteases on discharging isolated cnida and Hidaka [Advances in Comparative and Environmental Physiology, 15, 45-76 (1993)] discussed various agents which can trigger cnida discharge.

Alternatively, discharge may be effected by hydration with a water-based composition such as saline or water, thereby opening channels in skin and enhancing delivery of a subsequent topically applied agent.

Compositions of the present invention may, if desired, be presented in a pack, such as an FDA approved kit, which may contain one or more unit dosage forms containing the active ingredient. The pack may, for example, comprise metal or plastic foil. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. For example, the present teachings also contemplate use of the pharmaceutical compositions described herein e.g., comprising scopolamine for the treatment of motion sickness or as atropine (see Table 1, above).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Cnidocysts can Serve as a Tool for the Delivery of the Muscarinic Receptor Antagosist, Scopolamine

Materials and Methods

Cnidocysts Isolation and Formulation

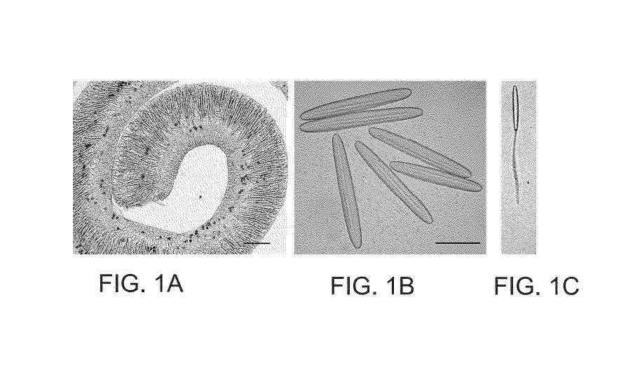

Cnidocysts were isolated from the filament acontia of Aiptasia diaphana based on their high density and high stability in salts as described previously (1, 2) (FIGS. 1A-C). Briefly, filaments were incubated in 1M sodium citrate, followed by two centrifugations in 70% percoll gradients. The isolated cnidocysts were washed with decreasing concentrations of NaCl (1M to 0.2M) and freeze-dried. The purified cnidocysts were kept dry until use, or formulated in an anhydrous gel consisting of 2% hydroxyl propyl cellulose in absolute ethanol.

Scopolamine Porcine Study:

To demonstrate systemic delivery by isolated cnidocysts, the potent muscarinic receptor antagonist, scopolamine, was selected. This naturally occurring alkaloid is one of the most effective single agents used to prevent motion sickness and was among the first drugs to be incorporated into a patch form for transdermal delivery. However its slow incremental transdermal diffusion demands patch application up to 8 hours in advance of need (7). As the cnidocysts system activity is immediate, a more rapidly detectable plasma levels is expected.

The study was performed on seven clinically normal white Landrance pigs, weighing approximately 20 kg each, under controlled conditions, according to the guidelines for testing the efficacy of veterinary medical products (Volume VII of The Rules Governing Medicinal Products in the European Union).

Four animals were treated with the cnidocyst gel and scopolamine, and three control animals received only the gel and scopolamine. The backs of the animals were gently shaved 24 h before the experiments. The 15 cm² area of application was rinsed with 70% ethanol, and a foam ring was applied to limit the application site. Test and control gel preparations were applied on each pig's back skin Immediately thereafter 5% scopolamine HBr solution was added and 5 minutes later the sites were rinsed with double distilled water and gently wiped to remove the applied product. Twelve blood samples were collected from each animal via the antebrachial cephalic sinus, at the following time points; immediately prior to dosing, and post dosing plus, 0.5 hr, 1 hr, 1.5 hr, 2 hr, 3 hr, 4 hr, 6 hr, 8 hr, 10 hr, 12 hr and 24 hr and the scopolamine pharmacokinetic parameters were calculated for each animal (6) (Table 2, below). Scopolamine concentrations were determined using a validated LC-MS/MS(+) method with a low limit of quantification of 10.4 ng/L (3). The HPLC was equipped with Merck LiChrospher 100 RP select B, 5 μm (4×75 mm) column and MS/MS Sciex API 4000 detector. Pharmacokinetic evaluation results of Cmax, Tmax, the half-life elimination time (t1/2) and the area under the concentration curve (AUC) results were calculated using Winnonlin (Pharsight) pharmacokinetic software. No abnormal or adverse physiological effects were observed in the pigs.

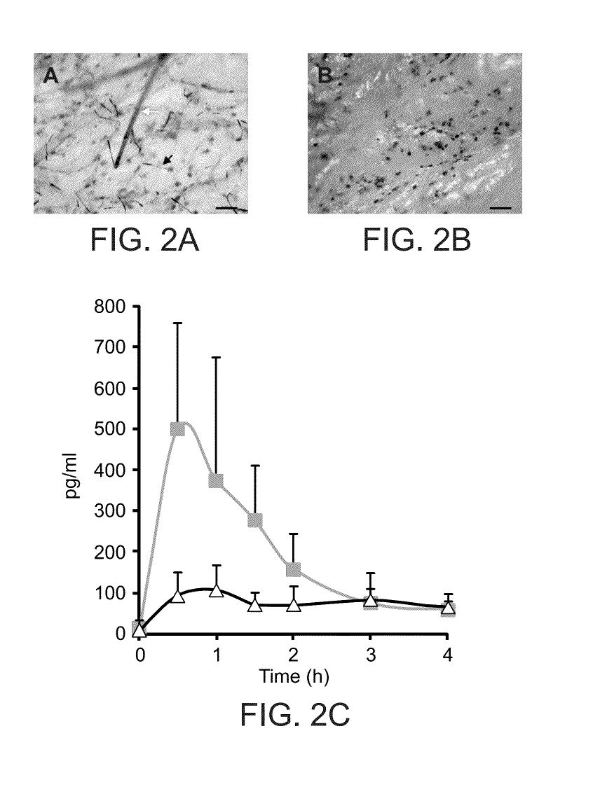

Cnidocysts-recipient animals showed up to five times higher peak plasma concentration (Cmax) compared to the control animals, 500±258 pg/ml and 107±61 pg/ml, respectively (FIG. 2C). Additionally, the time to maximal concentration (Tmax) was significantly shorter in the test versus the control animals, 0.5±0 versus 2.7±1.5 hours, respectively, and the average areas under the concentration curve (AUC₀₋₂₄) for the tested formulation were calculated to be approximately twice those of the control group. Although, the injection system is applied in topical formulation, it has a short application time and provides rapid accumulation in the plasma. These characteristics are similar to a subcutaneous injection with the convenience of a topical application.

TABLE 2
Calculated pharmacokinetic parameters of scopolamine
after 5-minute topical applications in pigs

| Pig No. | | | | | | | | |
|---|---|---|---|---|---|---|---|---|
| Test-cnidocyst preparation | | | | Control | | | | |
| 7A | 6A | 5A | 4A | 3A | 2A | 1A | Units | Parameter |
| 0.5 | 0.5 | 0.5 | 0.5 | 1 | 4 | 3 | hours | Tmax |
| 486.6 | 239.5 | 855.3 | 419.5 | 148.4 | 98.4 | 155 | {pcg/ml} | Cmax |
| 7.1 | 4.8 | 5.6 | 5.2 | 9.7 | 6.8 | 4.4 | hours | t½ |
| 1155.3 | 970.1 | 2488.9 | 1973.0 | 816.1 | 911.5 | 1191.5 | hrX{pcg/ml} | AUC₀₋₂₄ |
| 1289.9 | 1093.1 | 2628.7 | 2320.8 | 946.5 | 1028.7 | 1316.9 | hrX{pcg/ml} | AUC_{INF} |

### Example 2

### Cnidocysts can Serve as a Tool for the Delivery of the Muscarinic Receptor Antagonist, Atropine

The objective of this study was to determine the plasma kinetics of Atropine Sulfate, following a single topical administration of Atropine Sulfate Gel concomitantly applied with cnidocysts in comparison to intramuscular (IM) injection of Atropine Sulfate Solution, in the porcine model. Atropine plasma concentrations were determined using an HPLC/MS/MS method.

Materials and Methods

Cnidocysts Isolation and Formulation

Cnidocysts were isolated from the filament acontia of Aiptasia diaphana based on their high density and high stability in salts as described previously (1, 2) (FIGS. 1A-C). Briefly, filaments were incubated in 1M sodium citrate, followed by two centrifugations in 70% percoll gradients. The isolated cnidocysts were washed with decreasing concentrations of NaCl (1M to 0.2M) and freeze-dried. The purified cnidocysts were kept dry until use, or formulated in an anhydrous gel consisting of 2% hydroxyl propyl cellulose in absolute ethanol. 10% Atropine (Sigma) were formulated in a hydrous gel consisting of 1.7% hydroxyl propyl cellulose in DDW.

Atropine Porcine Study:

The current study was carried out to determine the plasma kinetics of Atropine, following a single topical administration of Atropine Sulfate Gel concomitantly applied with cnidocysts, in comparison to intramuscular (IM) injection of Atropine (Sigma) Sulfate Solution, to male Sinclair mini swine 6-7 months of age, 20.8-22.6 Kg at study initiation. Blood samples were collected prior to each dosing and at predetermined time points (5, 10, 15, 30, 45 min & 1, 2, 4, 12, and 24 hrs) following each dosing session for subsequent analysis of Test Items.

Dosing

The study included 3 dosing sessions as detailed in Table 3 below. Blood samples were collected prior to each dosing and at predetermined time points (5, 10, 15, 30, 45 min and 1, 2, 4, 12, and 24 hrs) following each dosing session for subsequent analysis of Test Items.

TABLE 3

| Administrated | | | | | | |
|---|---|---|---|---|---|---|
| Atropine Dose | Administrated | Atropine conc. (%) | Route of | | | |
| (mg/kg) | Atropine dose (mg) | in the formulation | Adm. | Session | Adm. Date | Pig # |
| 0.05 | 1.06 | 5 | *IM | 1 | 30/11/2009 | 1461 |
| 17.70 | 400 | 20 | topical | 2 | 07/12/2009 | |
| 17.47 | 400 | 20 | **topical | 3 | 14/12/2009 | |
| 4.81 | 100 | 5 | topical | 1 | 30/11/2009 | 1483 |
| 0.05 | 1.10 | 0.2 | IM | 2 | 07/12/2009 | |
| 0.05 | 1.13 | 0.2 | IM | 1 | 07/12/2009 | 1485 |
| 0.05 | 1.18 | 0.2 | IM | 2 | 14/12/2009 | |
| 19.80 | 400 | 20 | topical | 1 | 07/12/2009 | 1498 |
| 18.43 | 400 | 20 | topical | 2 | 14/12/2009 | |

*Pig not sedated
**Only atropine sulfate gel

Results

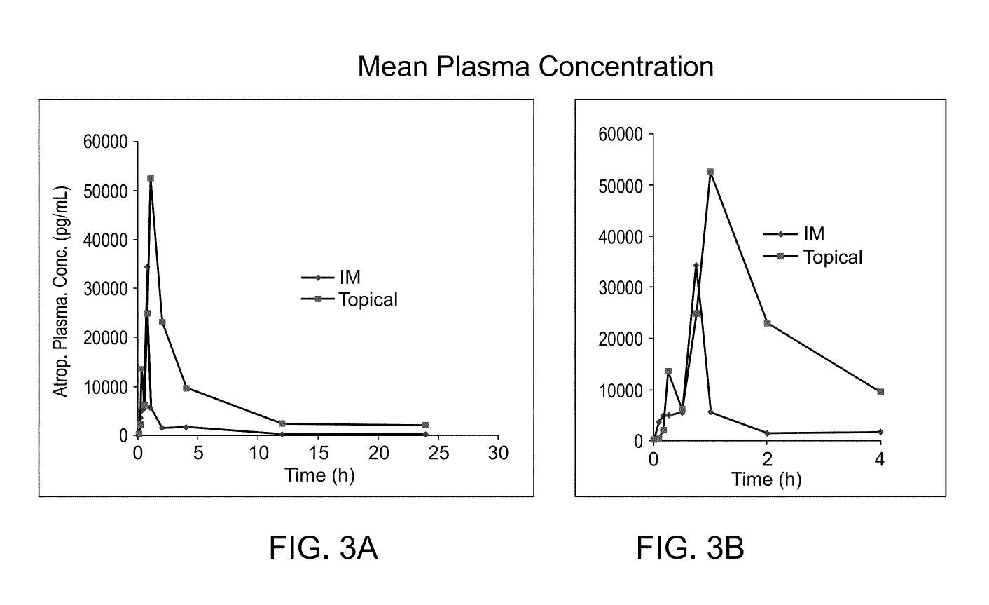

The mean plasma concentration-time curve of Atropine, is depicted for each route of administration in FIGS. 3A-B. Cumulative Value (CV) % of atropine concentrations at each time point is listed in Table 4 below.

TABLE 4

| 24 | 12 | 4 | 2 | 1 | 0.75 | 0.5 | 0.25 | 0.17 | 0.08 | Time |
|---|---|---|---|---|---|---|---|---|---|---|
| 131.4 | 112.9 | 59.9 | 27.6 | 32.5 | 105.7 | 16.2 | 18.3 | 46.3 | 55.4 | CV % IM |
| 144.3 | 96.4 | 113.2 | 98.9 | 103.7 | 148.9 | 42.5 | 76.9 | 43.6 | *131.2 | CV % Topical |

*n = 2 (time point 5 min obtained in pig-1498-S1-topical, was not taken into account)

This study shows a drastic enhancement of the bioavailability of atropine after topical administration using cnidocysts facilitated topical administration (see FIGS. 3A-B).

As compared to the IM administration the relative bioavailability of atropine after topical administration with adjunct was about 1.5%. This value depicts the amount of atropine in the plasma/the amount of atropine topically applied, thereby supporting bioavailability.

### CONCLUSION

This study that was conducted with a small number of subjects (mini-pigs) shows a drastic enhancement of the bioavailability of atropine after topical administration when cnidocysts were added to the formulation: the AUC normalized to the dose was about 8 fold higher when the adjunct was added to the formulation.

As compared to the IM administration the relative bioavailability of atropine after topical administration with adjunct (cnidocysts) was about 1.5%.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Other References are Cited Throughout the Application

### References and Notes

1. M. R. Prausnitz, R. Langer, Nat Biotech 26, 1261 (2008).
2. G. Cevc, U. Vierl, J. Controlled Release 141, 277 (2010).
3. C. N. David et al., Trends Genet 24, 431 (2008).
4. S. Szczepanek, M. Cikala, C. N. David, J Cell Sci 115, 745 (2002).
5. T. Nüchter, Benoit M., Engel U., Ozbek S., Holstein T W, Current Biology 16, R316 (2006).
6. Materials and methods are available as supporting material on Science Online
7. U. D. Renner, R. Oertel, W. Kirch, Ther Drug Monit 27, 655 (2005).
S1. A. Lotan, L. Fishman, Y. Loya, E. Zlotkin, Nature 375, 456. (1995).
S2. P. G. Greenwood, I. M. Balboni, C. Lohmann, Comp Biochem Physiol A Mol Integr Physiol 134, 275 (2003).
S3. R. Oertel, K. Richter, U. Ebert, W. Kirch, J Chromatogr B 750, 121 (2001)
S4. T. Lotan, in: Modified Release Drug Delivery Technology, 2^{nd} edition, Hadgraft J., Rathbone M. and Lane M. (eds) (2008).

What is claimed is:

1. A method of treating motion sickness in a subject in need thereof, the method comprising applying to a skin surface of the subject scopolamine and stinging cells or stinging capsules, said stinging cells or capsules being, upon discharge, capable of delivering said scopolamine into the subject in a systemic manner, so as to treat said motion sickness in the subject.

2. The method of claim 1, wherein said scopolamine and stinging cells or stinging capsules are in different formulations.

3. The method of claim 2, wherein said scopolamine and stinging cells or stinging capsules are in a two-component composition.

4. The method of claim 1, wherein said scopolamine is administered following said administering of said stinging cells or stinging capsules.

5. The method of claim 1, wherein said stinging cells or capsules are from Nematostella vectensis.
